(19) United States
(12) Patent Application Publication
(10) Pub. No.: US 2023/0008831 A1
(43) Pub. Date: Jan. 12, 2023

(54) TWO-STEP AUTO-INJECTION APPARATUS

(71) Applicant: Suzhou Hengrui Hongyuan Medical Technology Co., Ltd., Suzhou (CN)

(72) Inventors: Lin HUANG, Suzhou (CN); Pu SUN, Suzhou (CN); Can SUN, Suzhou (CN)

(73) Assignee: Suzhou Hengrui Hongyuan Medical Technology Co., Ltd., Suzhou (CN)

(21) Appl. No.: 17/784,098

(22) PCT Filed: May 12, 2020
(86) PCT No.: PCT/CN2020/089782

§ 371 Date: Jun. 9, 2022

(30) Foreign Application Priority Data
Feb. 24, 2020 (CN) 202010114315.4

(51) Int. Cl. A61M 5/20; A61M 5/32; A61M 5/315

(52) U.S. Cl. CPC A61M 5/20; A61M 5/3202; A61M 5/31515; A61M 2205/581

(57) ABSTRACT

The present invention provides a two-step auto-injection apparatus consisting of a housing, a protective sleeve cylinder, a pre-filled injection assembly and a drive feedback apparatus, wherein the pre-filled injection assembly is loaded into the housing, and the protective sleeve cylinder covers a needle of the pre-filled injection assembly, so that the protective sleeve cylinder directly contacts an injecting portion and the function of injection can be realized by pressing to trigger the drive feedback apparatus during use, thereby facilitating quick operation with a needle tip hidden in the whole process; the drive feedback apparatus of a piston in a needle cylinder is driven to combine a release mechanism with an ejection mechanism and a feedback mechanism, and the release mechanism is triggered by pressing the protective sleeve cylinder, so that the ejection mechanism is released, and the ejection mechanism interacts with the feedback mechanism in a form of collision, for example, to generate a sound signal and/or a haptic signal in the moment of release for prompting a patient to begin an injection; when the ejection mechanism finishes movement, the ejection mechanism interacts again with the feedback mechanism in the form of collision, for example, to generate the sound signal and/or the haptic signal for promoting the patient to finish the injection.

### BACKGROUND OF THE INVENTION

### Field of the Invention

The present invention relates to the technical field of injection apparatus design, in particular, to a two-step auto-injection apparatus.

Description of the Prior Art

With the development of science and technology and social progress, higher requirements have been put forward for the use of injection apparatuses, and the injection device is expected to be simple to operate. During the injection, there are feedbacks in multiple dimensions (e.g., audition, vision, haptic), which facilitate the patients to control the entire injection process, so as to reduce their anxiety. It is necessary to ensure that the nudity of the needle tip before and after the injection is prevented from causing fear to the user. The injection apparatus should also have anti-touch and/or anti-secondary use and other functions, while higher requirements are put forward for the miniaturization, portability, ease of use and economy of the product. How to realize the above functions and meet the market demands has attracted more and more attention of engineering and technical personnel.

The existing injector has the following disadvantages:

1. At present, in most cases, such products use electric motors and so on to provide power for the motion mechanisms, which requires energy consumption and high cost of use, thereby being easy to cause environmental pollution; the connection with a variety of transmission mechanisms will cause the overall large volume of the product, which results in certain requirements on the use of the installation space and operating space, and disability of being used in extreme conditions such as in a very small space.

2. At present, for the product designed with feedbacks in multiple dimensions, the auditory feedback and the visual feedback mostly rely on electronic devices, so that the product needs to have a power supply apparatus. If the product is in the environment with strong electromagnetic interference, it cannot achieve its due functions. The power supply apparatuses such as batteries are susceptible to environmental influences, and easy to aging. It is difficult to guarantee the stability, reliability and long-term storage of the whole apparatus when used in complex environments. Meanwhile, in order to be able to monitor the motion status of the mechanism in real time, members such as a sensor are required to feedback signals and also the signal transmission is required, so that the product is easy to be interfered with by the strong electromagnetic environment, thereby disabling the feedback functions of the product.

3. At present, most of such auto-injection products in the market are not convenient in operation, and usually require operation steps of more than two, which requires an unlocking step, resulting in complex operation processes and poor compliance of the patient.

4. The product requires a design for the related mechanism to prevent the product from being in secondary touch and use. Currently, most products adopt a solution of inserting limiting parts such as a limiting pin after use; in this way, in most cases, the situation of missing installation and missing the limiting parts may occur for the user, and this design method is often contrary to human factors engineering design and not in line with the development of the times.

5. In order to achieve auto-injection function, the existing product mostly adopt structural designs such as a snap buckle, wherein actions such as releasing and locking are performed by the elastic deformation of the parts; this design requires the structures such as the snap buckle to be in an unreleased state, and the state is a pressed and deformed state, which does not facilitate transportation and long-term storage while being greatly affected by the environment; this design is prone to cause plastic deformation and loses its due elasticity when in use, thereby being unable to be snapped into the due positions and causing failure of the product.

6. In the existing product, the feedback is performed by the sound generated by mechanical collision, and the pushing force required for the collision and injection is powered by an injection spring, which may cause situations such as resistance and uneven distribution of the force, and cause changes in the injection time and the injection amount, thereby resulting in low treatment efficacy.

### SUMMARY OF THE INVENTION

In view of the problems in the background, the present invention provides a two-step auto-injection apparatus, which includes a housing, a protective sleeve cylinder disposed in the housing, a pre-filled injection assembly and a drive feedback apparatus, wherein one end of the housing is a proximal end, and the other opposite end of the housing is a distal end;

the pre-filled injection assembly includes a needle cylinder, wherein one end of the needle cylinder is provided with a needle, the other end of the needle cylinder is provided with a piston, and the needle cylinder is filled with a drug liquid; the needle cylinder is mounted at an end in the housing close to the proximal end, and the needle at least partially protrudes out of the housing;

the drive feedback apparatus includes an ejection mechanism, a release mechanism and a feedback mechanism, wherein the ejection mechanism is connected with the piston, the release mechanism is used to release the ejection mechanism, and the ejection mechanism pushes the piston to move after being released; the feedback mechanism is used to generate a sound signal and/or a haptic signal at the beginning and end of the movement of the ejection mechanism to indicate the beginning and end of the movement of the ejection mechanism;

the protective sleeve cylinder is disposed at the proximal end of the housing, with one end extending into the housing to contact the release mechanism and be capable of moving relative to the housing and the other end protruding out of the housing; the protective sleeve cylinder covers the needle, with an end portion provided with a passage for the needle to protrude;

the protective sleeve cylinder is pressed on an injecting portion, and the protective sleeve cylinder moves from the proximal end to the distal end relative to the housing, wherein when the protective sleeve cylinder moves, the needle protrudes out of the passage to puncture into the injecting portion while the protective sleeve cylinder pushes the release mechanism to trigger the ejection mechanism, so that the ejection mechanism pushes the piston to move towards a side of the needle and the piston pushes the drug liquid to be injected into the injecting portion from the needle.

Preferably, a guide limit assembly is disposed between the end of the protective sleeve cylinder extending into the housing and an inner wall of the housing.

Preferably, the guide limit assembly includes a sliding groove disposed on the protective sleeve cylinder, and a limit boss disposed on the inner wall of the housing, the limit boss being disposed in the sliding groove; when the protective sleeve cylinder moves relative to the housing, the limit boss moves along the sliding groove, and the limit boss is limited after the limit boss moves until the sliding groove moves.

Preferably, the apparatus further includes an injector protection cap, wherein the injector protection cap covers the proximal end of the housing, the end portion of the protective sleeve cylinder is located in the injector protection cap, and the injector protection cap is detachably connected with the proximal end of the housing.

Preferably, the pre-filled injection assembly further includes a needle protection cap, and the needle protection cap covers the needle and is detachably connected with the needle cylinder.

Preferably, the injector protection cap is connected with the needle protection cap, and the injector protection cap drives the needle protection cap to be detached from the needle cylinder when being detached from the housing.

Preferably, the injector protection cap is detachably connected with the needle protection cap by a snap joint assembly.

Preferably, the snap joint assembly includes a bulking hook structure disposed on an inner side of the injector protection cap and a bulking port structure disposed on an outer side of the needle protection cap, and the bulking hook structure is bulked in the bulking port structure for realizing connection.

Preferably, the inner wall of the housing is provided with an inner hole boss, the needle cylinder and the drive feedback apparatus are arranged on both sides of the inner hole boss, and an end of the needle cylinder facing towards the distal end abuts against the inner hole boss.

Preferably, the ejection mechanism includes a guide tube and a pushing rod co-axially disposed in the guide tube, and a first energy storage member axially storing energy is disposed between the pushing rod and the guide tube; a first limiting structure is disposed between the pushing rod and the guide tube; an end of the pushing rod facing towards the proximal end protrudes out of the guide tube to be co-axially connected with the piston;

the release mechanism comprises a release sleeve cylinder co-axially sleeved on an outer side of the guide tube, and the release sleeve cylinder is provided with a second limiting structure; the protective sleeve cylinder extends into one end of the housing to contact the release sleeve cylinder; when the ejection mechanism is not released, the second limiting structure cooperates with the first limiting structure to position the pushing rod in the guide tube; when the protective sleeve cylinder axially moves to drive the release sleeve cylinder to axially move, the first limiting structure is separated from the second limit structure, and the pushing rod is pushed to move axially from the distal end to the proximal end under the action of the first energy storage member, thereby releasing the ejection mechanism.

Preferably, the feedback mechanism includes a feedback ring co-axially sleeved at an outer side of the pushing rod, and a second energy storage member for storing energy circumferentially is disposed between the feedback ring and the pushing rod; an outer wall of the feedback ring is provided with a first convex portion, and an inner wall of the guide tube is provided axially with a first guide groove for the first convex portion to slide; the first guide groove is provided internally with a beginning feedback portion and an end feedback portion; when the pushing rod begins and finishes the movement, the first convex portion passes through the beginning feedback portion and the end feedback portion respectively, the second energy storage member drives the first convex portion to strike a side wall of the first guide groove to generate the sound signal and/or the haptic signal for indicating the beginning and end of the movement of the system.

Preferably, the first limiting structure includes a second convex portion disposed on an outer wall of the pushing rod and a second guide groove disposed on the inner wall of the guide tube, and the second convex portion is located in the second guide groove.

Preferably, the second limiting structure includes a third convex portion disposed on the release sleeve cylinder, the guide tube is provided axially with a third guide groove penetrating through a side wall of the guide tube, and the third convex portion is located in the third guide groove;

the second guide groove is disposed adjacent to the third guide groove, and a side of the second guide groove is communicated circumferentially with the third guide groove; the side wall in the guide tube is further provided with a fourth guide groove communicated axially with the third guide groove;

when the ejection mechanism is not released, a side of the third convex portion abuts against a side of the second convex portion in the second guide groove to limit the second convex portion circumferentially; the second guide groove and an end of the second convex portion facing towards the proximal end form shapes of a slope that are matched with each other, and abut against each other to form a limit on the second convex portion axially;

when the release sleeve cylinder moves from the proximal end to the distal end, the third convex portion moves to be separated from the second convex portion, and the second convex portion slides into the third guide groove and the fourth guide groove sequentially under the action of the first energy storage member, the second convex portion moving along the fourth guide groove.

Preferably, the pushing rod is provided with two second convex portions that are arranged symmetrically, and the guide tube is provided with two second guide grooves correspondingly;

the release sleeve cylinder is provided with two third convex portions that are arranged symmetrically, and the guide tube is provided with two third guide grooves and two fourth guide grooves correspondingly.

Preferably, the first energy storage member is of a spring structure that is disposed co-axially with the pushing rod;

the distal end of the guide tube is provided with an end cap, and the energy storage member has one end connected with the pushing rod and the other end connected with the end cap; and initially, the first energy storage member is in an energy storage state.

Preferably, a side of the first guide groove is in a shape of a three-segmented ladder, and transition positions of adjacent ladders form the beginning feedback portion and the end feedback portion respectively.

Preferably, the first guide groove includes a distal end groove segment, an intermediate groove segment and a proximal end groove segment that are connected with each other sequentially, a groove width of the distal end groove segment is smaller than a groove width of the intermediate groove segment, and the groove width of the intermediate groove segment is smaller than a groove width of the proximal end groove segment;

when the ejection mechanism is not released, the first convex portion abuts against a side wall of the distal end groove segment under the action of the second energy storage member; the release sleeve cylinder is pushed to trigger the pushing rod to move axially, and the first convex portion falls into the intermediate groove segment from the distal end groove segment; when falling into the intermediate groove segment, the second energy storage member drives the feedback ring to rotate, so that the first convex portion strikes onto a side wall of the intermediate groove segment and generates the sound signal and/or the haptic signal to indicate the release of the ejection mechanism, and then the pushing rod begins to move;

the release sleeve cylinder continues to move from the distal end to the proximal end, and the first convex portion moves along the intermediate groove segment; after a movement stroke of the pushing rod is finished, the first convex portion falls into the proximal end groove segment from the intermediate groove segment; when falling into the proximal end groove segment, the second energy storage member drives the feedback ring to rotate, so that the first convex portion strikes onto a side wall of the proximal end groove segment and generates the sound signal and/or the haptic signal to indicate the end of the movement of the pushing rod.

Preferably, the feedback ring is provided with two first second convex portions that are arranged symmetrically, and the guide tube is provided with two first guide grooves correspondingly.

Preferably, the second energy storage structure is of a torsion spring structure and is sleeved on the pushing rod co-axially, and the torsion spring structure has one end connected with the feedback ring and the other end connected with the pushing rod; when the injection is not performed, the second energy storage structure is in the energy storage state.

Preferably, the apparatus further includes a self-locking assembly that is used to lock the release sleeve cylinder with the pushing rod after the movement of the pushing rod ends.

Preferably, the self-locking assembly includes:

a third energy storage member disposed between the release sleeve cylinder and the guide tube, wherein when the release sleeve cylinder moves from the proximal end to the distal end, the third energy storage member performs the energy storage;

a first lock catch member disposed on the proximal end of the release sleeve cylinder;

a second lock catch member disposed on the pushing rod;

when the movement of the pushing rod is finished, a force applied to the protective sleeve cylinder is canceled, a force applied to the release sleeve cylinder disappears, and the release sleeve cylinder is pushed to move from the distal end to the proximal end and the first lock catch member is engaged with the second lock catch member to form a self-locking under the action of the third energy storage member; meanwhile, the release sleeve cylinder pushes the protective sleeve cylinder to move from the distal end to the proximal end, and the needle is retracted into the protective sleeve cylinder.

Preferably, the first lock catch member is a fourth convex portion disposed on a side wall in the proximal end of the release sleeve cylinder, and a side of the fourth convex portion is in a shape of slope so that an end of the fourth convex portion close to the proximal end is narrower than an end close to the distal end;

the first convex portion forms the second lock catch member;

an outer wall of the proximal end of the guide tube is provided with a fifth guide groove, and the fourth convex portion is located in the fifth guide groove; an end portion of the proximal end of the first guide groove penetrates through an end portion of the proximal end of the fifth guide groove radially;

after the movement of the pushing rod is finished, the first convex portion moves to the end portion of the proximal end of the first guide groove, and is partially located in the fifth guide groove; the release sleeve cylinder moves from the distal end to the proximal end, and pushes the first convex portion to move circumferentially to be separated from the side wall of the first guide groove when passing through the fourth convex portion, while the first energy storage member performs the energy storage; the fourth convex portion crosses the first convex portion to be hooked on an end of the convex portion, while the first convex portion strikes the side wall of the first guide groove again under the action of the first energy storage member and generating the sound signal and/or the haptic signal to indicate the completion of the self-locking of the pushing rod.

Preferably, the third energy storage member is of a spring structure, and the third energy storage member is sleeved on an outer side of the release sleeve cylinder co-axially; and the third energy storage member has one end connected with the release sleeve cylinder and the other end connected with the pushing rod.

Preferably, a position on the housing corresponding to the needle cylinder is provided with an observation window for observing a forward process of the piston.

Due to the adoption of the above technical solutions, the present invention, as compared with the prior art, has the following advantages and active effects:

1. The present invention provides a two-step auto-injection apparatus consisting of a housing, a protective sleeve cylinder, a pre-filled injection assembly and a drive feedback apparatus, wherein the pre-filled injection assembly is loaded into the housing, and the protective sleeve cylinder covers a needle of the pre-filled injection assembly, so that the protective sleeve cylinder directly contacts an injecting portion and the function of injection can be realized by pressing to trigger the drive feedback apparatus during use, thereby facilitating quick operation with a needle tip hidden in the whole process and mitigating anxiety and panic of the user;

with the drive feedback apparatus used to drive a piston in a needle cylinder and a design of effectively combining a release mechanism with an ejection mechanism and a feedback mechanism, the release mechanism is triggered by pressing the protective sleeve cylinder, so that the ejection mechanism is released, and the ejection mechanism interacts with the feedback mechanism in the form of collision, for example, to generate a sound signal and/or a haptic signal in the moment of release for prompting a patient to begin an injection; when the ejection mechanism finishes the movement, the ejection mechanism interacts again with the feedback mechanism in the form of collision, for example, to generate the sound signal and/or the haptic signal for promoting the patient to finish the injection, so that the user may control the entire injection process, thereby further reducing the risk of misoperation and mitigating anxiety and panic.

2. The two-step auto-injection apparatus provided with the present invention consists of mechanical structures as a whole, which facilitates the simple assembly, and all parts (except the energy storage mechanism) are in a natural state without forced deformation after the product is assembled, wherein there are no restrictions on the conditions and years of use, thereby resulting in a high stable and reliable product; the entire system is assembled by way of embedding, a single part may have multiple functions at the same time, and the entire system has a compact structure with fewer parts, so that miniaturization and miniaturization may be realized, thereby greatly reducing the production cost;

in addition, for the entire system, various functions such as one-step release, automatic feedback, anti-retreat and self-locking may be achieved for the system only by pushing the protective sleeve cylinder, so that the operation steps are simple, and the risk of misoperation and missing operations is reduced for the user.

3. For the two-step auto-injection apparatus provided by the present invention, the side wall of the first guide groove with the shape of a ladder on the guide tube is collided by the first convex portion to generate sounds and/or vibrations, thereby prompting the user the current movement state of the entire system; this way of feeding back the movement state helps the operator to control the movement state of the product, is applicable to a wide range of people, and may be used in a variety of environments;

4. For the two-step auto-injection apparatus provided by the present invention, the overall release process of the drive feedback apparatus is completed in the form of circumferential changes in the track, i.e., transiting from the initial position (the second guide groove) to the movement track (the fourth guide groove), and the overall process does not require the structural elastic deformation in the traditional design to achieve release, thereby resulting in a highly reliable system.

5. For the two-step auto-injection apparatus provided by the present invention, through the provision of the self-locking assembly, the self-locking is achieved for the apparatus after the product is used, so that the personnel is prevented from being damaged and the environment is prevented from being polluted when the apparatus is in the secondary use or is discarded; also, the artificial non-essential removal may be prevented, which facilitate the effective protection for the internal parts.

### BRIEF DESCRIPTION OF THE DRAWINGS

Combined with the drawings, the above and other features and advantages of the present invention can be better understood through the detailed instructions described below, wherein:

FIG. 1 is a front view showing the state when the two-step auto-injection apparatus is not used according to the present invention;

FIG. 2 is an X-X cross-section view showing the state when the two-step auto-injection apparatus is not used according to the present invention;

FIG. 3 is a top view showing the state when the two-step auto-injection apparatus is not used according to the present invention;

FIG. 4 is a Y-Y cross-section view showing the state when the two-step auto-injection apparatus is not used according to the present invention;

FIG. 5 is a view showing the detachment of the two-step auto-injection apparatus according to the present invention;

FIG. 6 is a view of the two-step auto-injection apparatus when the needle protection cap is opened according to the present invention;

FIG. 7 is a view showing the state when the two-step auto-injection apparatus is used according to the present invention;

FIG. 8 is a front view showing the state when the two-step auto-injection apparatus is used according to the present invention;

FIG. 9 is a schematic structural view of the drive feedback apparatus according to the present invention;

FIG. 10 is a schematic structural view of the pushing rod according to the present invention;

FIG. 11 is a front view of the pushing rod according to the present invention;

FIG. 12 is a side view of the pushing rod according to the present invention;

FIG. 13 is an A-A cross-section view of the pushing rod according to the present invention;

FIG. 14 is a schematic structural view of the guide tube according to the present invention;

FIG. 15 is a front view one of the guide tube according to the present invention;

FIG. 16 is a front view two of the guide tube according to the present invention;

FIG. 17 is a side view of the guide tube according to the present invention;

FIG. 18 is a B-B cross-section view of the guide tube according to the present invention;

FIG. 19 is a C-C cross-section view of the guide tube according to the present invention;

FIG. 20 is a D-D cross-section view of the guide tube according to the present invention;

FIG. 21 is a schematic structural view of the release sleeve cylinder according to the present invention;

FIG. 22 is a front view of the release sleeve cylinder according to the present invention;

FIG. 23 is an E-E cross-section view of the release sleeve cylinder according to the present invention;

FIG. 24 is a schematic structural view of the feedback ring according to the present invention;

FIG. 25 is a front view of the feedback ring according to the present invention;

FIG. 26 is a schematic structural view of the end cover according to the present invention;

FIG. 27 is a radial cross-section view (from the proximal end to the distal end) of the drive feedback apparatus when being not in injection according to the present invention;

FIG. 28 is a view of the second limit structure limiting the first limit structure in the drive feedback apparatus when being not in injection according to the present invention;

FIG. 29 is an axial cross-section view of the pushing rod when being released according to the present invention;

FIG. 30 is a radial cross-section view (from the proximal end of the system to the distal end of the system) of the pushing rod when being released according to the present invention;

FIG. 31 is an axial cross-section view of the pushing rod when being ejected according to the present invention;

FIG. 32 is a radial cross-section view (from the proximal end of the system to the distal end of the system) of the pushing rod when being ejected according to the present invention;

FIG. 33 is an axial cross-section view of the pushing rod when being in movement according to the present invention;

FIG. 34 is a radial cross-section view (from the proximal end of the system to the distal end of the system) of the pushing rod when being in movement according to the present invention;

FIG. 35 is an axial cross-section view one of the pushing rod when finishing the movement according to the present invention;

FIG. 36 is a view of a process of resetting the release sleeve cylinder according to the present invention;

FIG. 37 is a radial cross-section view (from the proximal end of the system to the distal end of the system) of the pushing rod in the process of resetting according to the present invention;

FIG. 38 is a view of the release sleeve cylinder achieving self-locking according to the present invention;

FIG. 39 is a schematic structural view of the guide tube according to an alternative solution of the present invention;

FIG. 40 is a schematic view of the guide tube according to an alternative solution of the present invention;

FIG. 41 is an F-F cross-section view of the guide tube according to an alternative solution of the present invention;

FIG. 42 is a view one of the release sleeve cylinder achieving self-locking according to an alternative solution of the present invention;

FIG. 43 is a view two of the release sleeve cylinder achieving self-locking according to an alternative solution of the present invention.

### DESCRIPTION OF THE PREFERRED EMBODIMENTS

With reference to the drawings showing the embodiments of the present invention, the present invention will be described in more detail below. However, the present invention may be implemented in many different forms and should not be interpreted as being subject to the limitations of the embodiment proposed here. In contrast, these embodiments are proposed in order to achieve full and complete disclosure, and to enable those skilled in the art to fully understand the scope of the present invention. In these figures, for clarity, the dimensions and relative dimensions of the layers and areas may have been enlarged.

It should be noted that all the directional indications (such as up, down, left, right, front, back, . . . ) in the embodiments of the present invention are only used to explain the relative positional relationship, motion situation and the like between the components under a specific attitude (as shown in the drawings), and if the specific attitude changes, then the directional indications also change accordingly.

The present invention provides a two-step auto-injection apparatus, which is suitable for off-hospital treatment, patient self-injection and other occasions.

The two-step auto-injection apparatus consists of a housing, a protective sleeve cylinder, a pre-filled injection assembly and a drive feedback apparatus, wherein the pre-filled injection assembly is loaded into the housing, and the protective sleeve cylinder covers a needle of the pre-filled injection assembly, so that the protective sleeve cylinder directly contacts an injecting portion and the function of injection can be realized by pressing to trigger the drive feedback apparatus during use, thereby facilitating quick operation with a needle tip hidden in the whole process and mitigating anxiety and panic of the user;

with the drive feedback apparatus used to drive a piston in a needle cylinder and a design of effectively combining a release mechanism with an ejection mechanism and a feedback mechanism, the release mechanism is triggered by pressing the protective sleeve cylinder, so that the ejection mechanism is released, and the ejection mechanism interacts with the feedback mechanism in a form of collision, for example, to generate a sound signal and/or a haptic signal in the moment of release for prompting a patient to begin an injection; when the ejection mechanism finishes movement, the ejection mechanism interacts again with the feedback mechanism in the form of collision, for example, to generate the sound signal and/or the haptic signal for promoting the patient to finish the injection, so that the user may control the entire injection process, thereby further reducing the risk of misoperation and mitigating anxiety and panic.

Further explanation of the specific embodiments is given below:

### Embodiment 1

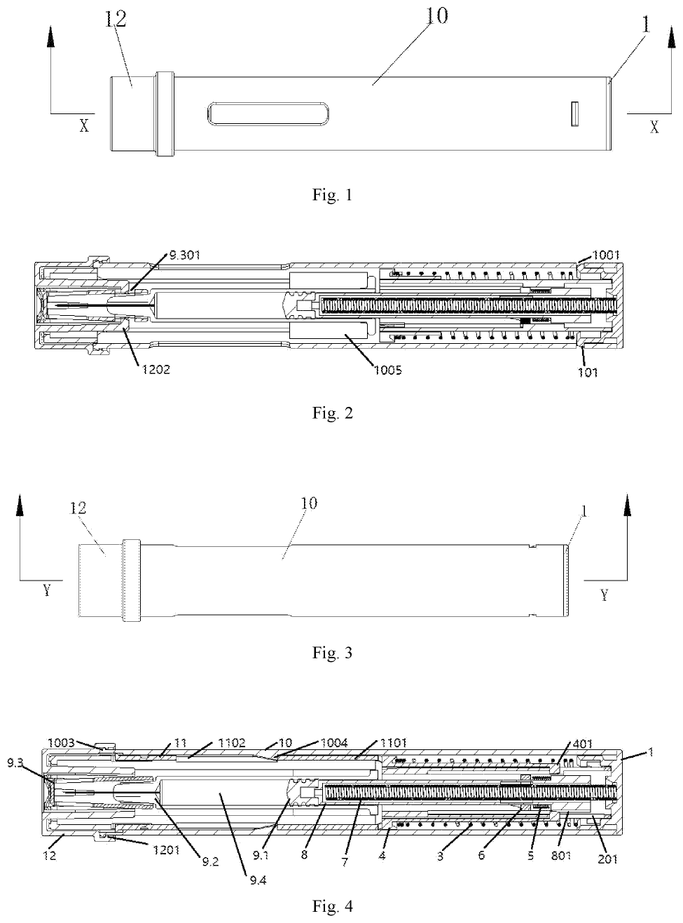

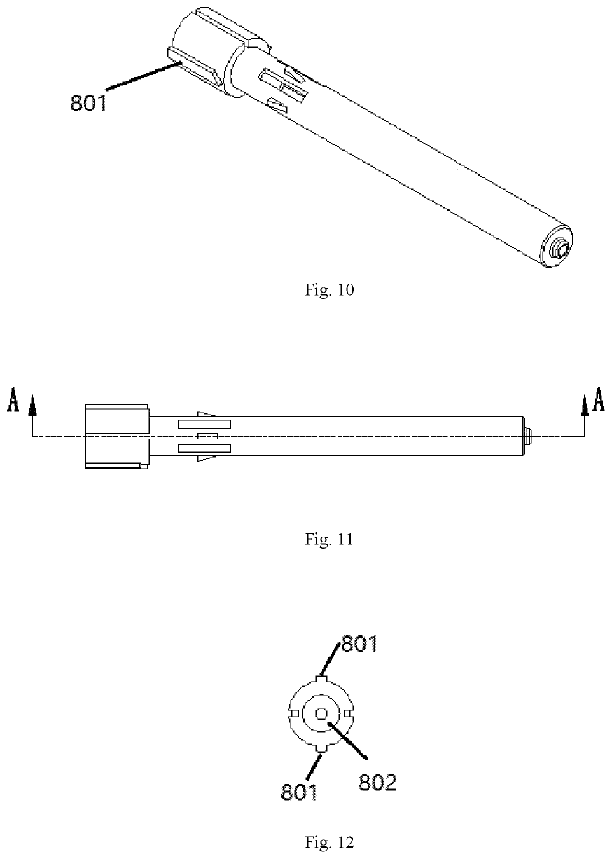

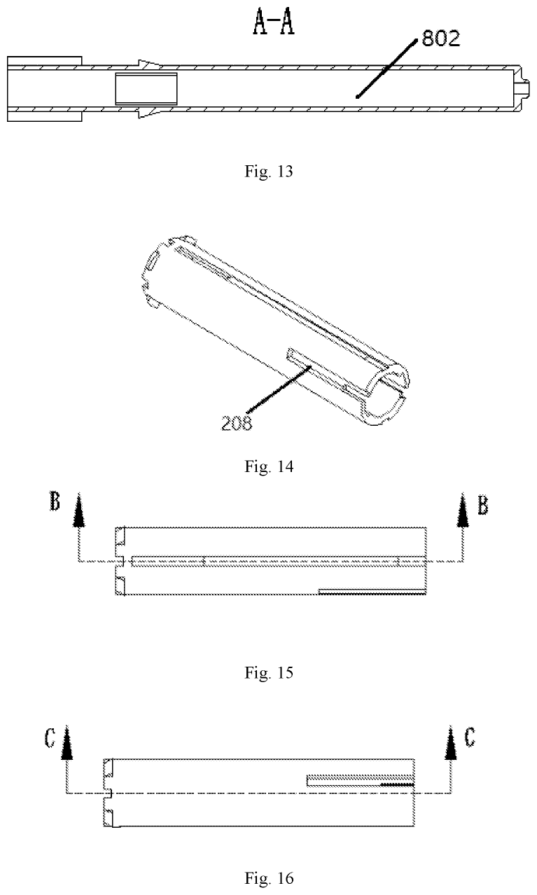

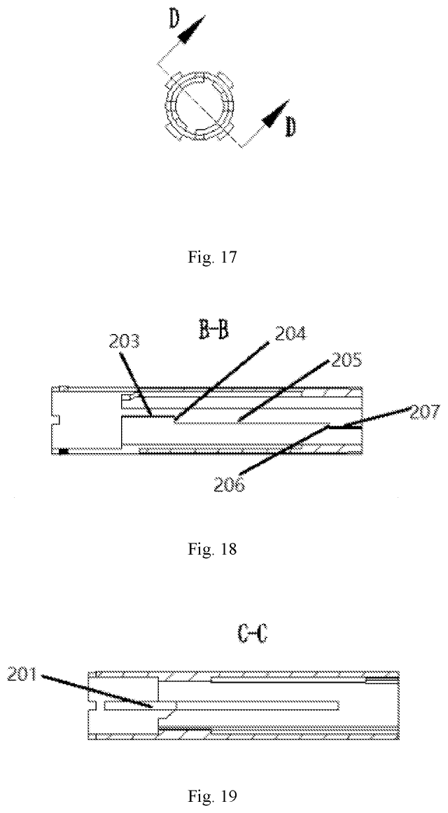

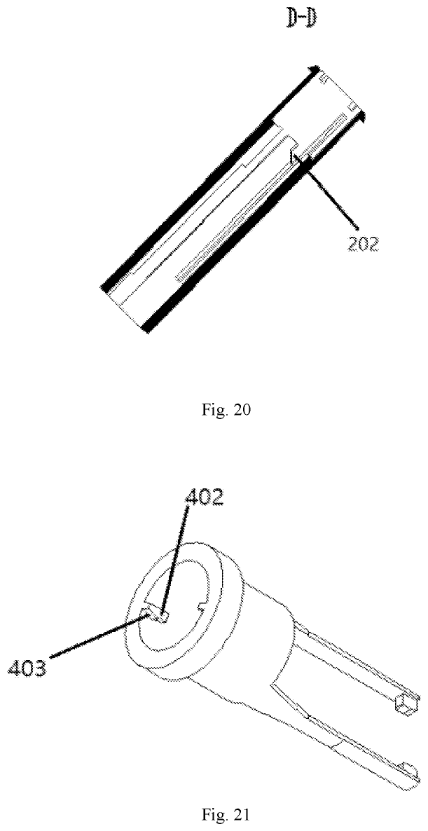

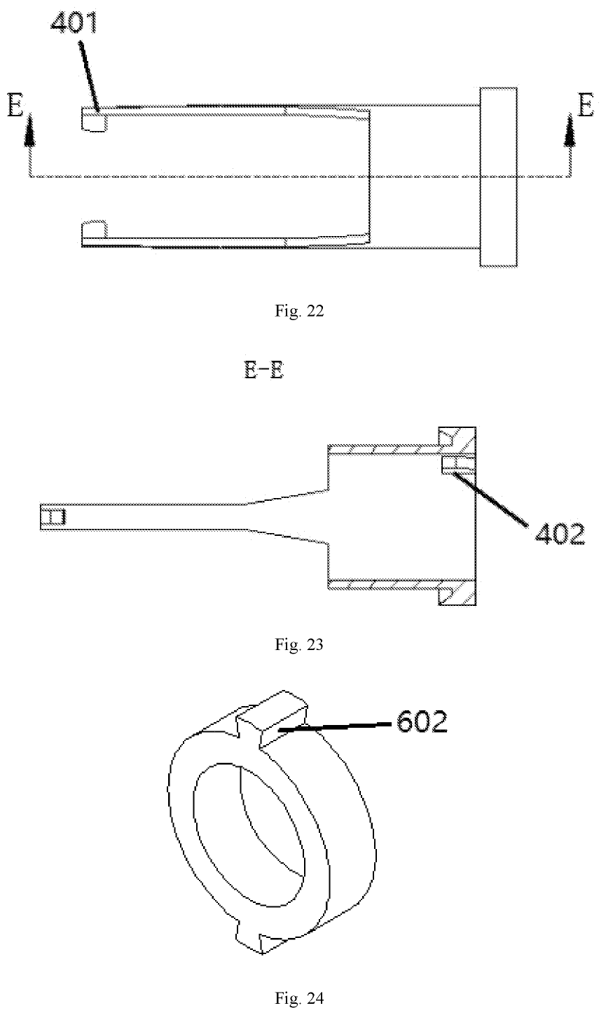

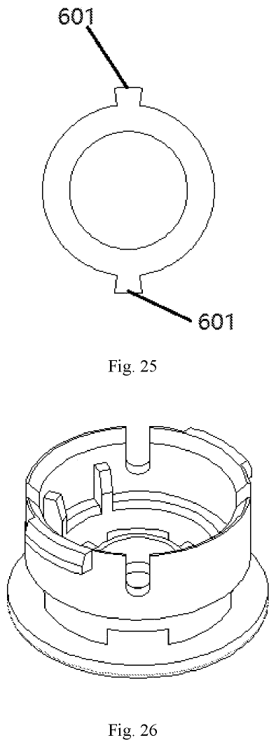

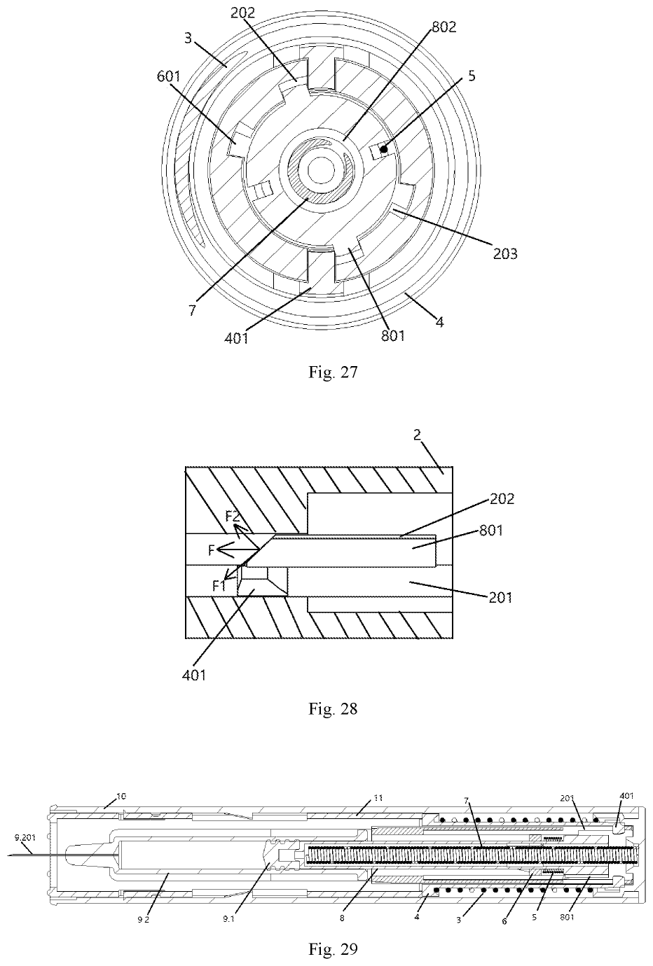

With reference to FIGS. 1 to 38, a two-step auto-injection apparatus provided by the present invention includes a housing 10, a protective sleeve cylinder 11 disposed in the housing 10, a pre-filled injection assembly 9 and a drive feedback apparatus, wherein one end of the housing 10 is defined as a proximal end and the other end of the housing is defined as a distal end; in other words, as shown in FIG. 1, the left end is defined as the proximal end and the right end is defined as the distal end.

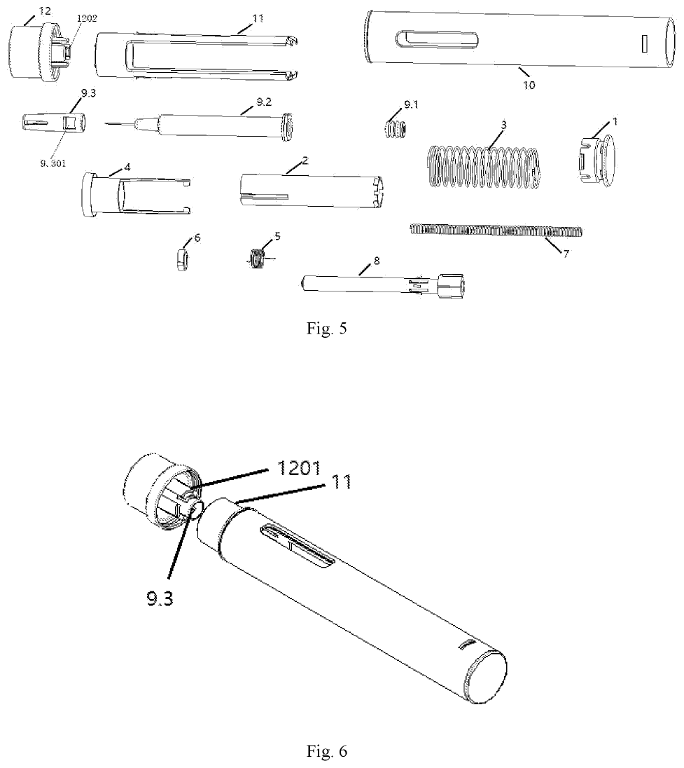

In the present embodiment, the housing 10 is of a cylindrical structure with openings at both ends, as shown in FIG. 5. Naturally, in other embodiments, the housing 10 may also be of a cuboid structure, etc., which is not limited here and may be adjusted according to actual needs.

In the present embodiment, with reference to FIGS. 1 to 5, the pre-filled injection assembly includes a needle cylinder 9.2, wherein one end of the needle cylinder 9.2 is provided with a needle 9.201 and the other end of the needle cylinder is provided with a piston 9.1, and the needle cylinder 9.2 is pre-filled with a drug liquid 9.4 to be injected. The needle cylinder 9.2 is mounted co-axially on an end of the housing 10 close to the proximal end, and the needle 9.201 is guaranteed to at least partially protrude out of the proximal end of the housing 10. The drive feedback apparatus is mounted on an end of the housing 10 close to the distal end for driving the piston 9.1 to move.

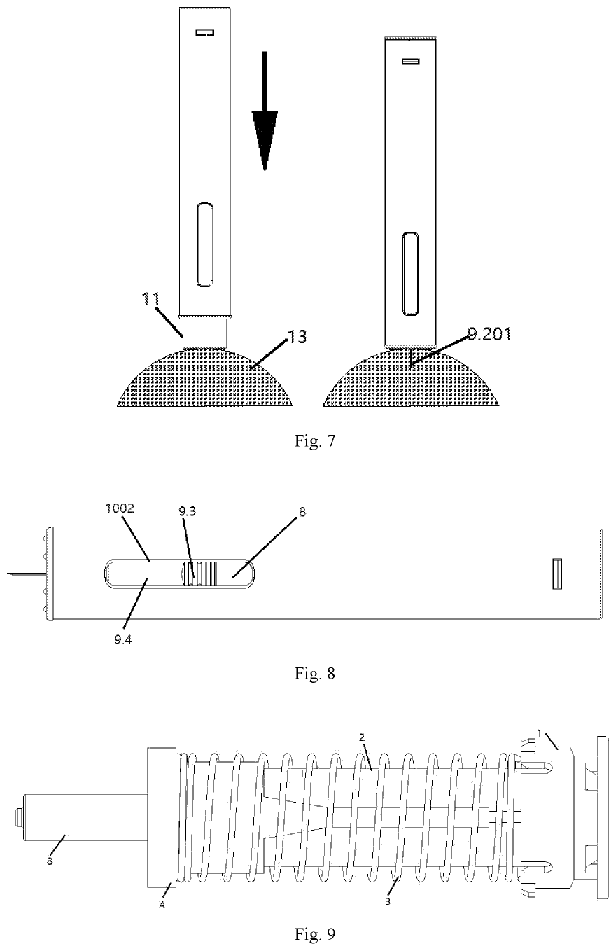

In the present embodiment, the protective sleeve cylinder 11 is disposed on the proximal end of the housing 10; one end of the protective sleeve cylinder 11 extends into the housing 10 and contacts the drive feedback apparatus as well as being able to move relative to the housing 10, and the other end of the protective sleeve cylinder protrudes out of the housing 110; the protective sleeve cylinder 11 covers the needle 9.201, and is provided with a passage for the needle to protrude; as shown in FIG. 7, the protective sleeve cylinder 11 is pressed on an injection portion, and the protective sleeve cylinder 11 moves from the proximal end to the distal end relative to the housing 10, wherein when the protective sleeve cylinder 11 moves, the needle 9.201 protrudes out of the passage to puncture into the injecting portion of an injected person while the protective sleeve cylinder 11 triggers the drive feedback apparatus, so that the drive feedback apparatus pushes the piston 9.1 to move towards a side of the needle and the piston 9.1 pushes the drug liquid to be injected into the injection portion from the needle 9.201.

Further, a guide limit assembly is disposed between the end of the protective sleeve cylinder 11 extending into the housing 10 and an inner wall of the housing 10. Specifically, as shown in FIG. 5, the end of the protective sleeve cylinder 11 extending into the housing 10 has two cantilevers 1101, wherein the two cantilevers 1101 are located between the housing 10 and the needle cylinder, and extend to the drive feedback apparatus for contacting therewith; the guide limit assembly includes a sliding groove 1102 disposed on the protective sleeve cylinder 11 and a limit boss 1004 disposed on the inner wall of the housing 10; naturally, in the process of the protective sleeve cylinder 11 extending into the housing 10, the protective sleeve cylinder is blocked by the limit boss 1004 while the cantilever 1101 converging inward, and after the protective sleeve cylinder crosses the limit boss 1004, the limit boss 1004 is embedded into the sliding groove 1102; when the protective sleeve cylinder 11 is pushed, the protective sleeve cylinder may move relative to the housing 10, and the limit boss 1004 moves along the sliding groove 1102, so that the displacement of the protective sleeve cylinder 11 is limited while ensuring the connection between the protective sleeve cylinder 11 and the housing 10.

Naturally, in other embodiments, the specific structural form of the guide limit assembly may be adjusted according to actual requirements, which is not limited here.

In the present embodiment, the inner wall of the housing 10 is provided with an inner hole boss 1005, wherein the needle cylinder 9.2 and the drive feedback apparatus are disposed on both sides of the inner hole boss 1005, an end of the needle cylinder facing towards the distal end is provided with a flange side, and the needle cylinder abuts against the inner hole boss 1005 through the flange side.

When the entire two-step auto-injection apparatus is mounted, the needle cylinder 9.2 is inserted from an opening of the proximal end of the housing 10 to abut the flange side of an end portion of the needle cylinder against the inner hole boss 1005, and the protective sleeve cylinder 11 is mounted on the proximal end of the housing 10, so that the needle cylinder 9.2 is limited between the protective sleeve cylinder 11 and the inner hole boss 1005; the drive feedback apparatus is inserted from an opening of the distal end of the housing 10, and then the distal end of the housing 10 is covered with an end cover 1, so that the drive feedback apparatus is limited between the end cover 1 and the inner hole boss 1005, thereby completing the assembly.

In the present embodiment, the two-step auto-injection apparatus further includes a needle protection cap 12, wherein the needle protection cap 12 is disposed at the proximal end of the housing 10, the protective sleeve cylinder 11 protrudes out of an end portion of the housing 10 to be located in the needle protection cap 12, and the needle protection cap 12 is detachably connected with the proximal end of the housing 10. In the present embodiment, through the provision of the needle protection cap 12, the protective sleeve cylinder 11 is protected, so as to prevent the injection due to the inadvertent touch of the protective sleeve cylinder 11 when is not used.

An outer wall of the proximal end of the housing 10 is provided with a lip 1003, and an inner wall of a connecting end of the needle protection cap 12 is provided with a ring-shaped protrusion 1201, wherein the needle protection cap 12 is sleeved on the proximal end of the housing 10, the ring-shaped protrusion 1201 is engaged to the lip 1003 to achieve detachable connection, and only the needle protection cap 12 is unplugged directly when the injection is required. Naturally, in other embodiments, the way in which the needle protection cap 12 is detachably connected with the housing 10 is not limited to the content mentioned above; for example, it may be achieved by way of threaded connection, etc.

In the present embodiment, the pre-filled injection assembly further includes a needle protection cap 9.3, the needle protection cap 9.3 is disposed on the needle and is detachably connected with the needle cylinder 9.2, and specifically, the needle protection cap 9.3 is directly engaged to the needle cylinder 9.2 through interference fit to achieve the detachable connection. In the present embodiment, through the provision of the needle protection cap 9.3, the needle cylinder is further protected, so as to prevent the inadvertent touch in case of being not used.

Further, the needle protection cap 9.3 is connected with the needle protection cap 12, wherein when the two are used for injection, the needle protection cap 12 is detached from the housing 10, while the needle protection cap 9.3 is driven to be detached from the needle cylinder 9.2 together. Specifically, an inner wall of the needle protection cap 12 is provided with an extension arm, and after the injector protection cap 12 is connected to the housing 10, the extension arm extends into the protective sleeve cylinder 11 from the passage of the end of the protective sleeve cylinder 11 that is used for the needle to protrude and is detachably connected to the needle protection cap 9.3 through a snap joint assembly. The snap joint assembly includes a buckling hook structure 1202 disposed on the extension arm and a buckling port structure 9.301 disposed on an outer side of the needle protection cap 9.3, and the buckling hook structure 1202 is buckled in the buckling port structure 9.301 to achieve connection. Naturally, in other embodiments, the way in which the needle protection cap 9.3 is connected with the needle protection cap 12 is not limited to the content mentioned above and may be adjusted according to actual situations, which are limited here.

In the present embodiment, as shown in FIG. 8, a position where the housing 10 corresponds to the needle cylinder 9.2 is provided with an observation window 1002 for observing a forward process of the piston, wherein the needle cylinder 9.2 is made from a transparent material, and the observation window 1002 may be an opening or a transparent portion, which is not limited here. In the present embodiment, through the provision of the observation window 1002, it is convenient for the user to observe the whole injection process, so as to achieve the effect of visual feedback.

In the present embodiment, with reference to FIG. 4 and FIGS. 9 to 26, the drive feedback apparatus includes an ejection mechanism, a release mechanism and a feedback mechanism.

The ejection mechanism includes a guide tube 2 and a pushing rod 8 co-axially disposed in the guide tube 2, and a first energy storage member 7 axially storing energy is disposed between the pushing rod 8 and the guide tube 2, an end of the pushing rod 8 facing towards the proximal end abutting against the piston 9.1; when the ejection mechanism is not released, the pushing rod 8 is positioned in the guide tube 2 through a first limiting structure; the release mechanism includes a release sleeve cylinder 4 co-axially sleeved on an outer side of the guide tube 2, the release sleeve cylinder 4 is provided with a second limiting structure, and the protective sleeve cylinder 11 extends into a cantilever 1101 in the housing 10 to abut against an end of the release sleeve cylinder 4 facing towards the proximal end; when the ejection mechanism is not released, the second limiting structure cooperates with the first limiting structure to position the pushing rod 8; when the injection is required, the protective sleeve cylinder 11 is axially pushed, the protective sleeve cylinder 11 further pushes the release sleeve cylinder 4 axially, the first limiting structure is removed from the second limiting structure, the second limiting structure fails, and the pushing rod 8 is pushed to move axially under the action of the first energy storage member 7, so that the ejection mechanism is triggered to be unlocked and the pushing rod begins to push the piston 9.1 to move;

the feedback mechanism includes a feedback ring 6 co-axially sleeved at an outer side of the pushing rod 8, and a second energy storage member 5 for storing energy circumferentially is disposed between the feedback ring 6 and the pushing rod 8, wherein the feedback ring 6, the second energy storage member 5 and the pushing rod form an integral mechanism and may move together; an outer wall of the feedback ring 6 is provided with a first convex portion 601, and an inner wall of the guide tube 2 is provided axially with a first guide groove for the first convex portion 601 to slide; the first guide groove is provided internally with a beginning feedback portion and an end feedback portion; when the pushing rod 8 begins and finishes the movement, the first convex portion 601 passes through the beginning feedback portion and the end feedback portion respectively, the second energy storage member 5 drives the first convex portion 601 to strike a side wall of the first guide groove to generate the sound signal and/or the haptic signal for indicating the beginning and end of the movement of the system.

In the present embodiment, the first energy storage member 7 is of a spring structure, which is disposed co-axially with the pushing rod 8; specifically, an end of the distal end of the guide tube 2 is covered with an end cover 1, and the end cover is mounted on the distal end of the guide tube 2 by way of snap buckling connection, etc.; an end of the pushing rod 8 facing towards the end cover 1 is provided co-axially with a mounting deep hole 802, and the first energy storage member 7 has one end extending into the mounting deep hole 802 and abutting against a bottom of the hole and has the other end abutting against the end cover 1. When being not in injection, i.e., when the pushing rod does not move, the first energy storage member 7 is in an energy storage state.

Naturally, in other embodiments, the first energy storage member may also be made from an elastic structure or materials with memory function, such as air cushions and other structures. The way in which the first energy storage member is mounted may be adjusted according to actual needs, which will not be limited here as long as the first energy storage member may store the energy axially relative to the pushing rod 8 and the guide tube 2.

In the present embodiment, the first limiting structure includes a second convex portion 801 disposed on an outer wall of the distal end of the pushing rod 8, and a second guide groove 202 disposed on an inner wall of the distal end of the guide tube 2, wherein the second convex portion 801 is located in the second guide groove 202; an end of the second convex portion 801 facing towards the proximal end is designed to be a first slope, an end of the second convex portion 801 close to the proximal end is designed to be a second slope, and the first slope presses the second slope tightly under the action of the first energy storage member.

The second limiting structure includes a third convex portion 401 disposed at the distal end of the release sleeve cylinder 4, specifically the distal end of the release sleeve cylinder 4 is provided with a distal end cantilever extending axially, and the third convex portion 401 is disposed on a side of an end of the distal end cantilever facing towards the guide tube 2, as shown in FIGS. 21 to 23; the distal end of the guide tube 2 is provided axially with a third guide groove 201 penetrating through a side wall of the guide tube 2, and the third convex portion 401 is located in the third guide groove 201; the second guide groove 202 is disposed adjacent to the third guide groove 201 circumferentially, and a side of the second guide groove 202 is communicated with the third guide groove 201 circumferentially; an inner side wall of the guide tube 2 is further provided with a fourth guide groove communicating with the third guide groove 201 axially, and the fourth guide groove is arranged to extend from the distal end to the proximal end.

When being not in injection, as shown in FIG. 4 and FIGS. 27 to 28, a side of the third convex portion 401 abuts against a side of the second convex portion 801 in the second guide groove 201, so as to limit the second convex portion 801 circumferentially; ends of the second guide groove 202 and the second convex portion 801 facing towards the proximal end are limited axially by tightly pressing the slopes; then, as shown in FIG. 28, the first energy storage member 7 exerts an axial pushing force F onto the second convex portion 801, wherein the pushing force F may be resolved into a component force Fl parallel to the slope exerted on the second convex portion 801 and a component force F2 perpendicular to the slope exerted on the second convex portion due to the contacting slopes;

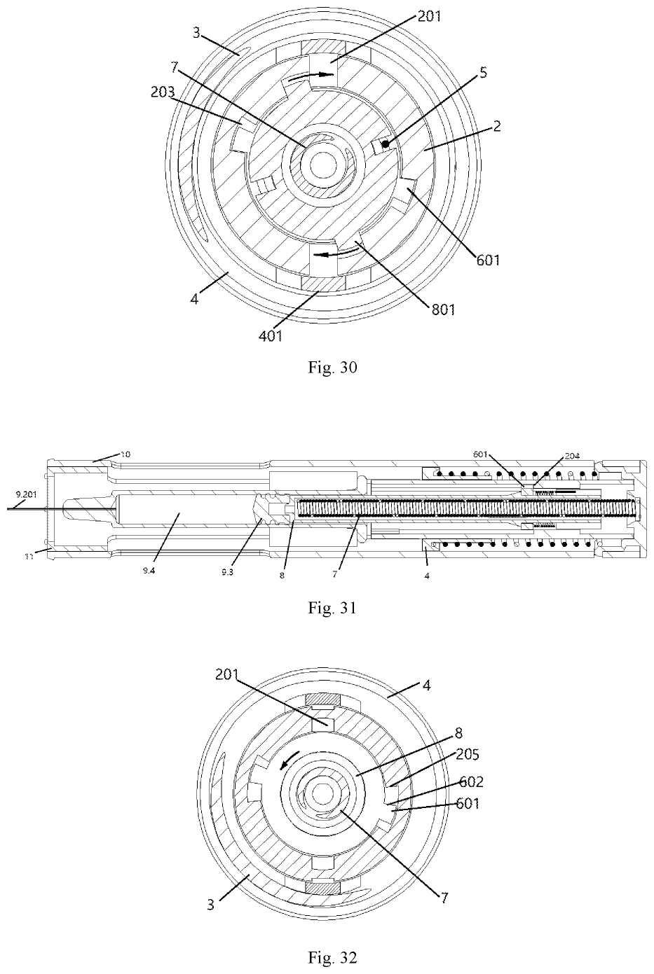

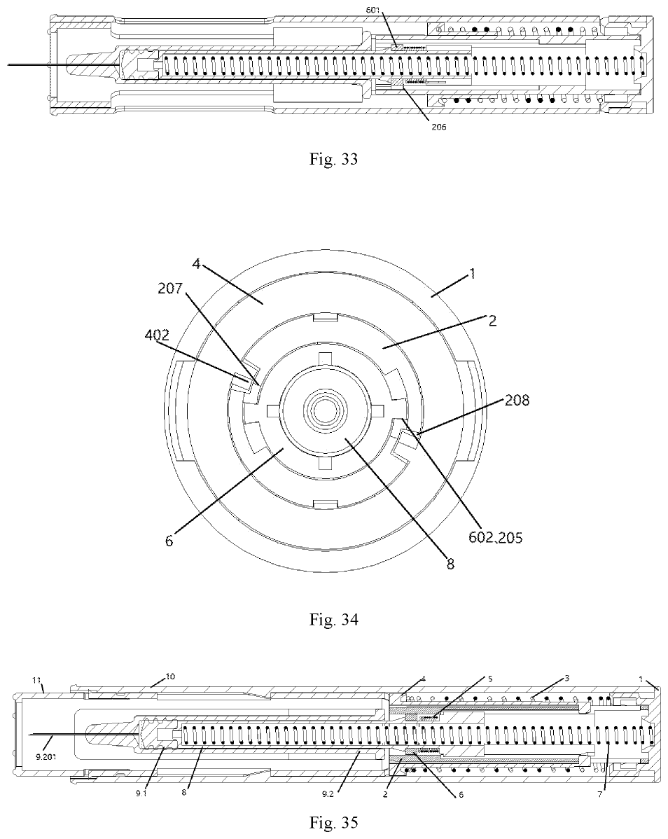

when the injection is required, the housing 10 is held by hands to press the protective sleeve cylinder 11 onto a targeted injection portion, and the protective sleeve cylinder 11 moves from the proximal end to the distal end, so that the release sleeve cylinder 4 is pushed to move axially from the proximal end to the distal end, the second convex portion 801 is driven to move towards the distal end along the second guide groove 201 and to cross the third convex portion 401, and the third convex portion 401 is failed to be limited circumferentially, as shown in FIGS. 29 to 30; then, under the action of Fl, the third convex portion 401 makes a spiral-like movement from the distal end to the proximal end (advancing towards the proximal end while rotating), and the third convex portion 401 first slides into the third guide groove 202 and then into the fourth guide groove under the action of the force F, as shown in FIGS. 31 to 32; in the subsequent movement of the pushing rod 8, the third convex portion 401 moves along the fourth guide groove, as shown in FIGS. 33 to 34.

In the present embodiment, the overall release process of the drive feedback apparatus is completed in the form of circumferential changes in the track, i.e., transiting from the initial position (the second guide groove) to the movement track (the fourth guide groove), and the overall process does not require the structural elastic deformation in the traditional design to achieve release, thereby resulting in a highly reliable system.

Further, in the present embodiment, preferably, the pushing rod 8 is provided with two second convex portions 801 that are disposed symmetrically, and the guide tube 2 is provided with two second guide grooves 202 correspondingly; the release sleeve cylinder 4 is provided with two third convex portions 401 that are disposed symmetrically, and the guide tube 2 is provided with two third guide grooves 201 and two fourth guide grooves correspondingly. In the present invention, through the provisions of two symmetrically-disposed first limiting structures and two symmetrically-disposed second limiting structures, the stability in limiting is ensured. Naturally, in other embodiments, the number and the arrangement way of the first limiting structure and the second limiting structure may be adjusted according to actual situations, e.g., only three circumferentially-disposed first limiting structures and three circumferentially-disposed second limiting structures are arranged uniformly, which is not limited here.

In the present embodiment, a side of the first guide groove is in a shape of ta hree-segmented ladder, and transition positions of adjacent ladders form the beginning feedback portion and the end feedback portion respectively.

Specifically, as shown in FIG. 18, the first guide groove is divided into a distal end groove segment 203, an intermediate groove segment 205 and a proximal end groove segment 207, wherein the distal end groove segment 203, the intermediate groove segment 205 and the proximal end groove segment 207 are connected in sequence from the distal end to the proximal end to form an integral first guide groove; a groove width of the distal end groove segment 203 is smaller than a groove width of the intermediate groove segment 205, and the groove width of the intermediate groove segment 205 is smaller than a groove width of the proximal end groove segment 207; a transition ladder 204 between the distal end groove segment 203 and the intermediate groove segment 205 forms a beginning feedback portion, and a transition ladder 206 between the intermediate groove segment 205 and the proximal end groove segment 207 forms an end feedback portion. The other side of the first guide groove is of a flat and straight shape, or of a ladder shape, or of other irregular shapes, which is not limited here.

When being not in injection, the second energy storage member 5 is in the energy storage state with a torque force stored therein, and the first convex portion 601 abuts against a side wall of the distal end groove segment 203 under the action of the torque force of the second energy storage member 5 to be in stationary; after the protective sleeve cylinder 11 pushes the release sleeve cylinder 4 to release the pushing rod, the pushing rod 8 drives the feedback ring 6 to move axially from the distal end to the proximal end together, and in the moment when the pushing rod 8 is released, the first convex portion 601 crosses the transition ladder 204 from the distal end groove segment 203 to fall into the intermediate groove segment 205; when being fallen into the intermediate groove segment 205, the feedback ring 6 is driven to rotate counterclockwise around an axis of the pushing rod 8 under the action of the torque force of the second energy storage member 5 as the groove width of the intermediate groove segment 205 is larger than that of the distal end groove segment 203, as shown in FIGS. 31 to 32; during the process, due to the large energy of the second energy storage member 5, the first convex portion 601 strikes a side wall of the intermediate groove segment 205 to make a strike sound of “click”, so that the user is prompted that the system is released to begin the movement by the sound signal. Naturally, in other embodiments, when the first convex portion 601 strikes the side wall of the intermediate groove segment 205, the strike sound of “click” is generated while causing a sense of vibration, so that the system is indicated to begin the movement by the sound signal and the haptic signal, or only the sense of the vibration is generated when the first convex portion 601 strikes the side wall of the intermediate groove segment 205 so that the user is prompted that the system begins the movement by the haptic signal.

After the pushing rod is released, the release sleeve cylinder 4 continues to move from the distal end to the proximal end, the first convex portion 601 moves along the intermediate groove segment 205; preferably, a length of the intermediate groove segment 205 is the movement stroke after the pushing rod 8 is released.

After the pushing rod 8 ends the movement stroke, i.e., after the injection is completed, the first convex portion 601 crosses the transition ladder 206 from the intermediate groove segment 205 to fall into the proximal end groove segment 207; when being fallen into the proximal end groove segment 207, the feedback ring 6 is driven to continue rotating counterclockwise around the axis of the pushing rod 8 under the action of the torque force of the second energy storage member 5 as the groove width of the proximal end groove segment 207 is larger than that of the intermediate groove segment 205, as shown in FIGS. 33 to 34; during the process, due to the large energy of the second energy storage member 5, the first convex portion 601 strikes a side wall of the proximal end groove segment 207 to make a striking sound of “click”, so that the user is prompted that the system ends the movement by the sound signal, as shown in FIG. 35. Naturally, in other embodiments, when the first convex portion 601 strikes the side wall of the proximal end groove segment 207, the striking sound of “click” is generated while causing a sense of vibration, so that the system is indicated to end the movement by the sound signal and the haptic signal, or only the sense of the vibration is generated when the first convex portion 601 strikes the side wall of the proximal end groove segment 207 so that the user is prompted that the system ends the movement by the haptic signal.

For the drive feedback apparatus provided by the present invention, the side wall of the first guide groove with the shape of ladder on the guide tube is collided by the first convex portion 601 to generate sounds and/or vibrations, thereby prompting the user the current movement state of the entire system; this way of feeding back the movement state helps the operator to control the movement state of the product, is applicable to a wide range of people, and may be used in a variety of environments.

In the present embodiment, the second energy storage structure 5 is preferably of a torsion spring structure and is sleeved on the pushing rod 8 co-axially; the torsion spring structure has one end connected with the feedback ring 6 and the other end connected to the pushing rod 8; specifically, in the present embodiment, the other end of the torsion spring structure is connected to the second convex portion 801. Naturally, in other embodiments, the second energy storage structure 5 may also be made from other elastic materials or materials with memory function, which is not limited here.

In the present embodiment, preferably, the feedback ring 6 is provided with two first convex portions 601 that are symmetrically disposed, and the guide tube 2 is provided with two first guide grooves correspondingly; through such provision, the stability and the reliability in feedback may be ensured. Naturally, in other embodiments, more than two first convex portions 601 and first guide grooves may also be arranged, which is not limited here.

In the present embodiment, the drive feedback apparatus further includes a self-locking assembly for locking the release sleeve cylinder 4 with the pushing rod 8 after the movement of the pushing rod 8 ends.

When the injector has a limit on the number of times for use (single use) or when the internal structure of the product needs to be protected after use, a design is required for the related mechanism to prevent the product from being in secondary touch and use. In the present embodiment, through the provision of the self-locking assembly, the above problems are solved.

Specifically, the self-locking assembly includes a third energy storage member 3 disposed between the release sleeve cylinder 4 and the guide tube 2, a first lock catch member disposed on the proximal end of the release sleeve cylinder 4 and a second lock catch member disposed on the pushing rod 8; when the release sleeve cylinder 4 moves from the proximal end to the distal end, the third energy storage member 3 stores the energy; after the movement of the pushing rod ends (after the injection is completed), i.e., when the first convex portion 601 strikes the side wall of the proximal end groove segment 207, the user acquires the sound signal and/or the haptic signal indicating the end of the movement of the system, and then releases the injection apparatus, so that the force of the protective sleeve cylinder 11 disappears, i.e., the external force exerted on the release sleeve cylinder 4 is canceled; since the energy is stored during the process of pushing the release sleeve cylinder 4 to move from the proximal end to the distal end, the third energy storage member 3 releases the energy to push the release sleeve cylinder 4 to move from the distal end to the proximal end after the external force is canceled, so that the first lock catch member is buckled with the second lock catch member for realizing self-locking, thereby self-locking the drive feedback apparatus. During this process, the release sleeve cylinder 4 may also push the protective sleeve cylinder 11 to move from the distal end to the proximal end, so that the needle 9.201 is retracted into the protective sleeve cylinder 11, the needle is hidden, and the self-locking of the drive feedback apparatus may prevent the retraction of the protective sleeve cylinder 11 to avoid the needle from protruding again.

Further, in the present embodiment, the first lock catch member is a fourth convex portion 402 disposed on an inner side wall of the proximal end of the release sleeve cylinder 4, and a side of the fourth convex portion 402 is of a slope shape to form a wedge-shaped surface 403, so that an end of the fourth convex portion 402 close to the proximal end is narrower than an end thereof close to the distal end, as shown in FIG. 21; the first convex portion 601 forms the second lock catch member.

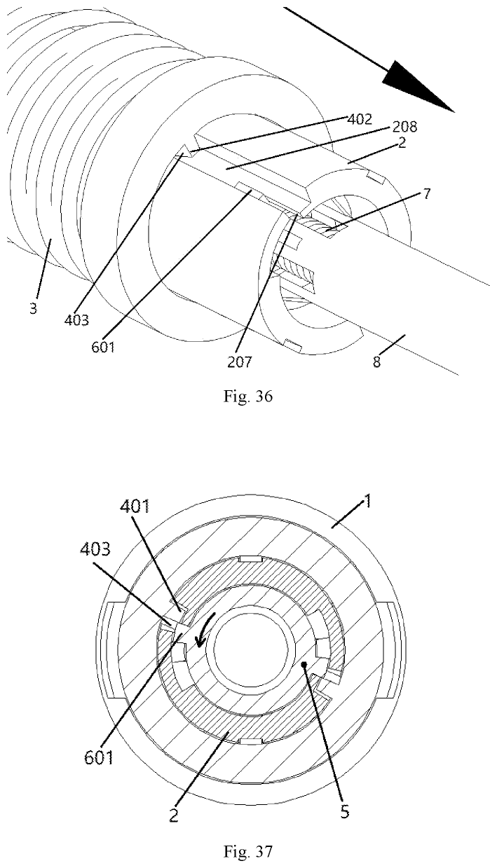

An outer wall of the proximal end of the guide tube 2 is provided with a fifth guide groove 208, and the fourth convex portion 402 is located in the fifth guide groove 208; further, an end portion of the proximal end of the first guide groove radially penetrates through an end portion of the proximal end of the fifth guide groove 208, i.e., the proximal end groove segment 207 partially penetrates through the fifth guide groove 208, and the first convex portion 601 falls into the proximal end groove segment 207 and then is partially located in the fifth guide groove 208, as shown in FIG. 36;

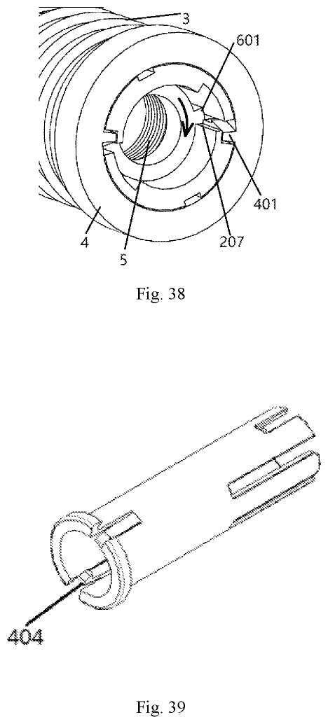

after the user acquires the sound signal and/or the haptic signal indicating the end of the movement of the system, the injection apparatus is released, so that the force of the protective sleeve cylinder 11 disappears, i.e., the external force exerted on the release sleeve cylinder 4 is canceled, the release sleeve cylinder 4 moves from the distal end to the proximal end to drive the fourth convex portion 402 to move from the distal end to the proximal end along the fifth guide groove 208, and then the first convex portion 601 is partially located in the second guide groove 208 and a gap is formed between a side surface of the first convex portion 601 and a side wall of the fifth guide groove 208; when the fourth convex portion 402 moves towards the first convex portion 601 and passes the gap, the wedge-shaped surface 403 contacts the first convex portion 601 and pushes the first convex portion 601 to rotate counterclockwise axially, as shown in FIG. 37, and in this process, the first convex portion 601 drives the feedback ring to convey counterclockwise and enable the second energy storage member 5 to store the energy; the fourth convex portion 402 continues to move towards the proximal end with the release sleeve cylinder 4, and after the fourth convex portion 402 crosses the first convex portion 601, the distal end of the fourth convex portion 402 is hooked to the proximal end of the first convex portion 601 to be locked, so that the release sleeve cylinder 4 is locked with the pushing rod 8, thereby realizing the locking of the entire injection apparatus; in this process, the first convex portion 601 rotates counterclockwise to strike again the side wall of the proximal end groove segment 207 under the action of the second energy storage member 5 to make a “click” sound for the third time, thereby prompting the user that the self-locking of the entire injection apparatus is completed by the sound signal, as shown in FIG. 38. Naturally, in other embodiments, when the first convex portion 601 strikes the side wall of the proximal end groove segment 207, the striking sound of “click” is generated while causing a sense of vibration, so that the self-locking of the system is indicated by the sound signal and the haptic signal, or only the sense of the vibration is generated when the first convex portion 601 strikes the side wall of the proximal end groove segment 207 so that the user is prompted the self-locking of the system by the haptic signal.

The self-locking assembly provided by the present embodiment directly realizes self-locking with the cooperation between the feedback ring on the feedback mechanism and the fourth convex portion 402 on the release sleeve cylinder, which is clever in design, simple in structure and has multiple purposes, thereby greatly simplifying the structure of the system; meanwhile, the self-locking of the apparatus is indicated by the sound signal and/or the haptic signal, so as to easy to control the movement state of the product for the operator, thereby being widely used in the population and in various environments.

The working principle of the two-step auto-injection apparatus provided by the present invention is further described as below, specifically:

when the injection apparatus is not in use, the drive feedback apparatus inside radially abuts against the second convex portion 801 through the third convex portion 401, thereby positioning the pushing rod in the guide tube, wherein the entire drive feedback apparatus is in a relatively stationary state;

when the injection is required, the injector protective cap 12 is unplugged while unplugging the needle protective cap 9.3, and the housing 10 is held by hands to abut the protective sleeve cylinder 11 against the injection portion and to press downward; then the protective sleeve cylinder 11 is pushed to move from the proximal end to the distal end to further push the release sleeve cylinder 4 to move from the proximal end to the distal end, and the release sleeve cylinder 4 drives the third convex portion 401 to be removed from the second convex portion 801; then, the second convex portion 801 is radially released, and the second convex portion 801, the pushing rod 8 and the feedback ring 6 make a spiral-like movement as a whole (i.e., displaced axially and circumferentially) under the action of the first energy storage member, so that the second convex portion 801 changes the track from the second guide groove to the fourth guide groove to realize releasing axially, thereby completing releasing the pushing rod 8; meanwhile, the feedback ring 6 axially moves to drive the convex portions to fall into the intermediate groove segment 205 from the distal end groove segment 203, and strikes the side wall of the intermediate groove segment 205 under the action of the second energy storage member to make a “click” sound, so as to prompt the user that the pushing rod begins the movement, i.e., the injection begins;

after the pushing rod is released, the pushing rod moves from the distal end to the proximal end under the action of the first energy storage member 7; again in the process, the first convex portion 601 moves along the intermediate groove segment 205 of the first guide groove, and the second convex portion 801 moves along the fourth guide groove, so as to realize guiding the axial movement of the pushing rod;

after the movement stroke of the pushing rod is completed, i.e., after the injection is completed, the first convex portion 601 just falls into the proximal end groove segment 207 from the intermediate groove segment 207, and strikes the side wall of the proximal end groove segment 207 under the second energy storage member to make a “click” sound for the second time, so as to indicate the user that the movement of the pushing rod ends, i.e., the injection is completed;

after the user receives the “click” sound of the second time, the user releases the injection apparatus, and the release sleeve cylinder 4 moves from the distal end to the proximal end under the action of the third energy storage member 3 to drive the fourth convex portion 402 to move along the fifth guide groove 208 while pushing the first convex portion 601 to rotate for cross the first convex portion 601 to be hooked to the first convex portion, so as to realize the locking; again in the process of pushing the first convex portion 601 to rotate and reset again, the first convex portion 601 strikes the side wall of the proximal end groove segment 207 again to make a “click” sound for the third time, so as to indicate the user that the self-locking of the apparatus is completed. During this process, the release sleeve cylinder 4 may also push the protective sleeve cylinder 11 to move from the distal end to the proximal end, so that the needle 9.201 is retracted into the protective sleeve cylinder 11, and the needle is hidden.

The two-step auto-injection apparatus provided by the present invention has the following advantages:

1. The entire injection apparatus is made up of mechanical structures, so that the assembly is simple, and each part (except the energy storage mechanism) is in a natural state without forced deformation after the product is assembled; the conditions of use and the service life are not limited, and the product has a high stability and reliability; and the present invention may be divided into subsystems (e.g., the drive feedback apparatus serves as a subsystem, the pre-filled injection apparatus serves as a subsystem, and the housing, the protective sleeve cylinder and the injector protective cap serve as a subsystem) provided to pharmaceutical enterprises to complete the final assembly, so as to ensure that the pre-filled injection apparatus (containing drug liquids) is assembled in subsequent steps, the probability of the overall scrapping of the product due to nonconformity of the remaining components is reduced, and the reduction of cost is greatly facilitated for the product enterprises;

2. In addition to visual feedback, the present invention may also achieve auditory and haptic feedback, so as to be easy to control the injection state of the product for the operator, to be applicable to a wide range of people and to be used in a variety of environments;

3. The product is easy to use with simple operation steps, and various functions such as automatic injection, feedback, anti-retreat and self-locking are achieved through two-step operation, thereby reducing the risk of misoperation of the user;

4. In the present invention, the power source of the feedback signal is not the same as that of the injection, which reduces blockade and resistance caused due to the uneven distribution of power during injection, and the feedback signal is more obvious, especially the feedback signal is not attenuated by the attenuation of the injection force when the injection is completed, so that the operator may better identify the feedback signal;

5. The entire injection apparatus is assembled in a nested way and the core components may have a variety of functions simultaneously; the entire product has a compact structure with fewer parts, so that miniaturization and miniaturization may be realized, thereby greatly reducing the production cost;

6. Before and after the injection apparatus is used, the needle may not bare out, thereby reducing the patients fear; the self-locking is achieved for the release mechanism after the product is used, so that the personnel is prevented from being damaged and the environment is prevented from being polluted when the apparatus is in the secondary use or is discarded; also, the artificial non-essential removal may be prevented, which facilitate the effective protection for the internal parts.

### Embodiment 2

The present embodiment is an adjustment based on Embodiment 1, wherein the self-locking assembly is adjusted in the present embodiment relative to Embodiment 1.

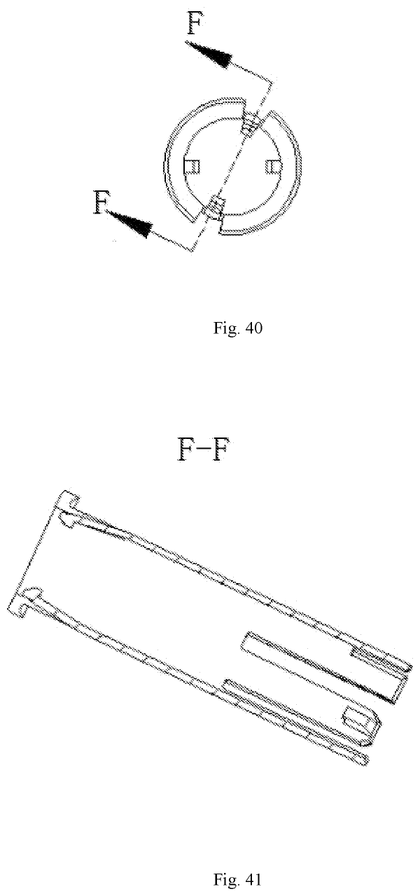

With reference to FIGS. 39 to 43, specifically, in the present embodiment, the first lock catch member in the self-locking assembly adopts an soft elastic tab 404, wherein the soft elastic tab 404 has one end connected to the release sleeve cylinder 4 and the other end arranged to extend to the proximal end and into the release sleeve cylinder 4, and an end portion of the other end of the soft elastic tab 404 is provided with a hooking portion; the guide tube 2 is provided with a passage groove for extending the soft elastic tab 404 into the guide tube 2, and the other end of the soft elastic tab 404 passes through a via to extend into the guide tube while extending towards the proximal end, the passage groove being communicated with the proximal end groove segment 207; the second lock catch member directly adopts the first convex portion 601, and slopes that are matched with each other are disposed on the first convex portion 601 and the hooking portion.

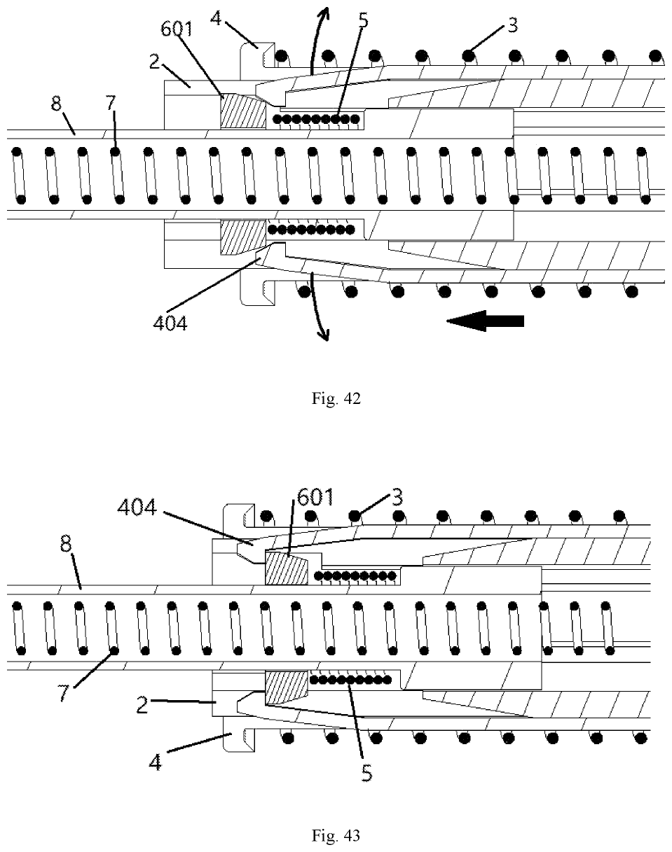

After the movement of the pushing rod is completed, the first convex portion 601 is located in the proximal end groove segment 207; the release sleeve cylinder 4 is released, the release sleeve cylinder 4 moves from the distal end to the proximal end under the action of the third energy storage member 3, and the slope of the hooking portion of the soft elastic tab 404 interacts along the slope of the first convex portion 601 and pushes the soft elastic tab 404 to expand outward when the hooking portion of the soft elastic tab 404 passes the first convex portion 601 so that the hooking portion of the soft elastic tab 404 crosses the first convex portion 601 successfully, as shown in FIG. 41; after the hooking portion of the soft elastic tab 404 crosses the first convex portion 601, the soft elastic tab 404 resets under the action of its own elasticity and allows the hooking portion to be hooked to the first convex portion 601 for realizing locking, thereby realizing self-locking of the system, as shown in FIG. 42.

Those skilled in the art should understand that the present invention may be realized in many other concrete forms without departing from its own spirit or scope. Although the embodiment of the present invention has been described, it should be understood that the present invention should not be limited to these embodiments, and those skilled in the art may make changes and modifications within the spirit and scope of the present invention as defined in the attached claims.

1-24. (canceled)

25. A two-step auto-injection apparatus, comprising a housing, a protective sleeve cylinder disposed in the housing, an injection assembly and a drive feedback apparatus, wherein one end of the housing is a proximal end, and the other opposite end of the housing is a distal end;
the drive feedback apparatus comprises an ejection mechanism, a release mechanism and a feedback mechanism, wherein the ejection mechanism is connected with the piston, the release mechanism is used to release the ejection mechanism; the feedback mechanism is used to generate a sound signal and/or a haptic signal at the beginning;
the ejection mechanism comprises a guide tube and a pushing rod co-axially disposed in the guide tube, and a first energy storage member axially storing energy is disposed between the pushing rod and the guide tube; a first limiting structure is disposed between the pushing rod and the guide tube; an end of the pushing rod facing towards the proximal end protrudes out of the guide tube to be co-axially connected with the piston;
the release mechanism comprises a release sleeve cylinder co-axially sleeved on an outer side of the guide tube, and the release sleeve cylinder is provided with a second limiting structure; the protective sleeve cylinder extends into one end of the housing to contact the release sleeve cylinder; when the ejection mechanism is not released, the second limiting structure cooperates with the first limiting structure to position the pushing rod in the guide tube; when the protective sleeve cylinder axially moves to drive the release sleeve cylinder to axially move, the first limiting structure is separated from the second limit structure, and the pushing rod is pushed to move axially from the distal end to the proximal end under the action of the first energy storage member, thereby releasing the ejection mechanism.

26. The two-step auto-injection apparatus according to claim 25, wherein the protective sleeve cylinder is pressed on an injecting portion, and the protective sleeve cylinder moves from the proximal end to the distal end relative to the housing, wherein when the protective sleeve cylinder moves, the needle protrudes out of the passage to puncture into the injecting portion while the protective sleeve cylinder pushes the release mechanism to trigger the ejection mechanism, so that the ejection mechanism pushes piston to move towards a side of the needle and the piston pushes the drug liquid to be injected into the injecting portion from the needle.

27. The two-step auto-injection apparatus according to claim 25, wherein a guide limit assembly is disposed between the end of the protective sleeve cylinder extending into the housing and an inner wall of the housing.

28. The two-step auto-injection apparatus according to claim 27, wherein the guide limit assembly comprises a sliding groove disposed on the protective sleeve cylinder, and a limit boss disposed on the inner wall of the housing, the limit boss being disposed in the sliding groove; when the protective sleeve cylinder moves relative to the housing, the limit boss moves along the sliding groove, and the limit boss is limited after the limit boss moves until the sliding groove moves.

29. The two-step auto-injection apparatus according to claim 25, wherein the first limiting structure comprises a second convex portion disposed on an outer wall of the pushing rod and a second guide groove disposed on the inner wall of the guide tube, and the second convex portion is located in the second guide groove.

30. The two-step auto-injection apparatus according to claim 25, wherein the second limiting structure comprises a third convex portion disposed on the release sleeve cylinder, the guide tube is provided axially with a third guide groove penetrating through a side wall of the guide tube, and the third convex portion is located in the third guide groove;
the second guide groove is disposed adjacent to the third guide groove, and a side of the second guide groove is communicated circumferentially with the third guide groove; the side wall in the guide tube is further provided with a fourth guide groove communicated axially with the third guide groove;
when the ejection mechanism is not released, a side of the third convex portion abuts against a side of the second convex portion in the second guide groove to limit the second convex portion circumferentially; the second guide groove and an end of the second convex portion facing towards the proximal end form shapes of a slope that are matched with each other, and abut against each other to form a limit on the second convex portion axially;
when the release sleeve cylinder moves from the proximal end to the distal end, the third convex portion moves to be separated from the second convex portion, and the second convex portion slides into the third guide groove and the fourth guide groove sequentially under the action of the first energy storage member, the second convex portion moving along the fourth guide groove.

31. The two-step auto-injection apparatus according to claim 25, wherein the pushing rod is provided with two second convex portions that are arranged symmetrically, and the guide tube is provided with two second guide grooves correspondingly;
the release sleeve cylinder is provided with two third convex portions that are arranged symmetrically, and the guide tube is provided with two third guide grooves and two fourth guide grooves correspondingly.

32. The two-step auto-injection apparatus according to claim 25, wherein the first energy storage member is of a spring structure that is disposed co-axially with the pushing rod;
the distal end of the guide tube is provided with an end cap, and the energy storage member has one end connected with the pushing rod and the other end connected with the end cap; and initially, the first energy storage member is in an energy storage state.

33. A two-step auto-injection apparatus, comprising a housing, a protective sleeve cylinder disposed in the housing, an injection assembly and a drive feedback apparatus, wherein one end of the housing is a proximal end, and the other opposite end of the housing is a distal end;
the drive feedback apparatus comprises an ejection mechanism, a release mechanism and a feedback mechanism, wherein the ejection mechanism is connected with the piston, the release mechanism is used to release the ejection mechanism, and the ejection mechanism pushes the piston to move after being released;
the ejection mechanism comprises a guide tube and a pushing rod co-axially disposed in the guide tube,
the feedback mechanism comprises a feedback ring co-axially sleeved at an outer side of the pushing rod, and a second energy storage member for storing energy circumferentially is disposed between the feedback ring and the pushing rod; an outer wall of the feedback ring is provided with a first convex portion, and an inner wall of the guide tube is provided axially with a first guide groove for the first convex portion to slide; the first guide groove is provided internally with a beginning feedback portion and an end feedback portion; when the pushing rod begins and finishes the movement, the first convex portion passes through the beginning feedback portion and the end feedback portion respectively, the second energy storage member drives the first convex portion to strike a side wall of the first guide groove to generate the sound signal and/or the haptic signal.

34. The two-step auto-injection apparatus according to claim 33, wherein the protective sleeve cylinder is pressed on an injecting portion, and the protective sleeve cylinder moves from the proximal end to the distal end relative to the housing, wherein when the protective sleeve cylinder moves, the needle protrudes out of the passage to puncture into the injecting portion while the protective sleeve cylinder pushes the release mechanism to trigger the ejection mechanism, so that the ejection mechanism pushes piston to move towards a side of the needle and the piston pushes the drug liquid to be injected into the injecting portion from the needle.

35. The two-step auto-injection apparatus according to claim 33, wherein a side of the first guide groove is in a shape of a three-segmented ladder, and transition positions of adjacent ladders form the beginning feedback portion and the end feedback portion respectively.

36. The two-step auto-injection apparatus according to claim 33, wherein the first guide groove comprises a distal end groove segment, an intermediate groove segment and a proximal end groove segment that are connected with each other sequentially, a groove width of the distal end groove segment is smaller than a groove width of the intermediate groove segment, and the groove width of the intermediate groove segment is smaller than a groove width of the proximal end groove segment;
when the ejection mechanism is not released, the first convex portion abuts against a side wall of the distal end groove segment under the action of the second energy storage member; the release sleeve cylinder is pushed to trigger the pushing rod to move axially, and the first convex portion falls into the intermediate groove segment from the distal end groove segment; when falling into the intermediate groove segment, the second energy storage member drives the feedback ring to rotate, so that the first convex portion strikes onto a side wall of the intermediate groove segment and generates the sound signal and/or the haptic signal to indicate the release of the ejection mechanism, and then the pushing rod begins to move;
the release sleeve cylinder continues to move from the distal end to the proximal end, and the first convex portion moves along the intermediate groove segment; after a movement stroke of the pushing rod is finished, the first convex portion falls into the proximal end groove segment from the intermediate groove segment; when falling into the proximal end groove segment, the second energy storage member drives the feedback ring to rotate, so that the first convex portion strikes onto a side wall of the proximal end groove segment and generates the sound signal and/or the haptic signal to indicate the end of the movement of the pushing rod.

37. The two-step auto-injection apparatus according to claim 33, wherein the feedback ring is provided with two first second convex portions that are arranged symmetrically, and the guide tube is provided with two first guide grooves correspondingly.

38. The two-step auto-injection apparatus according to claim 33, wherein the second energy storage structure is of a torsion spring structure and is sleeved on the pushing rod co-axially, and the torsion spring structure has one end connected with the feedback ring and the other end connected with the pushing rod; when the injection is not performed, the second energy storage structure is in the energy storage state.

39. A two-step auto-injection apparatus, comprising a housing, a protective sleeve cylinder disposed in the housing, an injection assembly and a drive feedback apparatus, wherein one end of the housing is a proximal end, and the other opposite end of the housing is a distal end;
the drive feedback apparatus comprises an ejection mechanism, a release mechanism and a feedback mechanism, wherein the ejection mechanism is connected with the piston, the release mechanism is used to release the ejection mechanism, and the ejection mechanism pushes the piston to move after being released;
the ejection mechanism comprises a guide tube and a pushing rod co-axially disposed in the guide tube,
the self-locking assembly comprises: a third energy storage member disposed between the release sleeve cylinder and the guide tube, wherein when the release sleeve cylinder moves from the proximal end to the distal end, the third energy storage member performs the energy storage;
a first lock catch member disposed on the proximal end of the release sleeve cylinder;
a second lock catch member disposed on the pushing rod;
when the movement of the pushing rod is finished, a force applied to the protective sleeve cylinder is canceled, a force applied to the release sleeve cylinder disappears, and the release sleeve cylinder is pushed to move from the distal end to the proximal end and the first lock catch member is engaged with the second lock catch member to form a self-locking under the action of the third energy storage member.

40. The two-step auto-injection apparatus according to claim 39, wherein the first lock catch member is a fourth convex portion disposed on a side wall in the proximal end of the release sleeve cylinder, and a side of the fourth convex portion is in a shape of slope so that an end of the fourth convex portion close to the proximal end is narrower than an end close to the distal end;
the first convex portion forms the second lock catch member;
an outer wall of the proximal end of the guide tube is provided with a fifth guide groove, and the fourth convex portion is located in the fifth guide groove; an end portion of the proximal end of the first guide groove penetrates through an end portion of the proximal end of the fifth guide groove radially;
after the movement of the pushing rod is finished, the first convex portion moves to the end portion of the proximal end of the first guide groove, and is partially located in the fifth guide groove; the release sleeve cylinder moves from the distal end to the proximal end, and pushes the first convex portion to move circumferentially to be separated from the side wall of the first guide groove when passing through the fourth convex portion, while the first energy storage member performs the energy storage; the fourth convex portion crosses the first convex portion to be hooked on an end of the convex portion, while the first convex portion strikes the side wall of the first guide groove again under the action of the first energy storage member and generating the sound signal and/or the haptic signal to indicate the completion of the self-locking of the pushing rod.

41. The two-step auto-injection apparatus according to claim 39, wherein the third energy storage member is of a spring structure, and the third energy storage member is sleeved on an outer side of the release sleeve cylinder co-axially; and the third energy storage member has one end connected with the release sleeve cylinder and the other end connected with the pushing rod.
